(19) United States
(12) Patent Application Publication
(10) Pub. No.: US 2009/0117597 A1
(43) Pub. Date: May 7, 2009

(54) Method for Measuring Heptoglobin Level in Blood Serum and Kit Therefor

(76) Inventors: Yu-Sam Kim, Seoul (KR); Kook-Jin Lim, Seoul (KR)

Correspondence Address: FISH & RICHARDSON PC, P.O. BOX 1022, MINNEAPOLIS, MN, 55440-1022, US

(21) Appl. No.: 11/719,439

(22) PCT Filed: Nov. 16, 2005
(86) PCT No.: PCT/KR2005/003882

§ 371 Date: Dec. 11, 2008

(30) Foreign Application Priority Data
Nov. 16, 2004 (KR) 10-2004-0093348

(51) Int. Cl. G01N 33/566

(52) U.S. Cl. 435/7.94; 436/501

(57) ABSTRACT

The present invention relates to a method for measuring heptoglobin level in blood serum by using antibodies for α and β subunits of heptoglobin, and a kit for immunodetection of heptoglobin which comprises the antibodies for α and β subunits of heptoglobin for measuring heptoglobin level in blood serum. A method for measuring heptoglobin level in blood serum of the present invention comprises the steps of: reacting blood serum of a subject with an antibody (αh antibody) which binds specifically to α subunit of a dimeric heptoglobin and an antibody (βh antibody) which binds specifically to β subunit of a dimeric heptoglobin; and, measuring level of proteins which react with the αh antibody and βh antibody in blood serum and have a molecular weight of more than 100 kDa. The method for measuring heptoglobin level in blood serum can be practically applied for the early diagnosis of diseases which disrupt erythrocytes, since only the level of normal heptoglobin except for monomelic and degraded hep-toglobins can be specifically measured by the said method.

### TECHNICAL FIELD

The present invention relates to a method for measuring heptoglobin level in blood serum and an immunodetection kit therefor, more particularly, to a method for measuring heptoglobin level in blood serum by using antibodies for α and β subunits of heptoglobin, and a kit for immunodetection of heptoglobin which comprises the antibodies for α and β subunits of heptoglobin for measuring heptoglobin level in blood serum.

### BACKGROUND ART

In normal cases, erythrocytes become senescent after 120 days, and degraded in bone marrow, liver and pancreas. However, rupture of erythrocytes owing to an attack of malaria or autoimmune disease frequently lead to the secretion of hemoglobin into blood. Hemoglobin is the most abundant metalloprotein in erythrocyte and plays a crucial role of oxygen-transport in body. Hemoglobin may exert a bad influence upon human body, since iron-containing heme group of the secreted hemoglobin produces a reactive oxygen species harmful to the cells. When hemoglobin is secreted abnormally as described, the secreted hemoglobin is degraded by leucocyte in a form of hemoblobin-heptoglobin (Hb-Hp) complex.

Heptoglobin is a macromolecular glycoprotein in blood serum of mammal, which is composed of two subunits, i.e., α subunit and β subunit. The reported molecular weights of the subunits have varied with the α subunit having been described as 9 and 16.5 kDa, and the β subunit having been described as 40 kDa. Heptoglobin is synthesized as a single chain and separated into N-terminal α subunit and C-terminal β subunit. α subunit and β subunit are linked by disulfide bond to form a heterodimer. In biological environments such as blood, heptoglobin binds to hemoglobin to form a stable Hb-Hp complex which is degraded by leucocyte.

When erythrocytes are disrupted in blood by a disease such as malaria, hemoglobins are secreted into blood serum and bound to heptoglobin, and forms Hb-Hp complex which is degraded by leucocyte. Thus, continuous disruption of erythrocytes may lead to decrease in heptoglobin level in blood serum. For the diagnosis of malaria, whose external symptom at an early stage of infection is not shown, a method for examining blood from a patient has been used for a while. But, a method of counting the numbers of erythrocytes has come into wide use presently, since the secreted hemoglobin is degraded rapidly. The method for counting the numbers of erythrocytes in human blood is, however, proven to be less satisfactory in a sense that it has a wide range of errors, and does not distinguish a normal from a malaria patient accurately.

As an approach of overcoming the said problem, a method for counting the numbers of heptoglobin in blood serum has been practiced for the diagnosis and prognosis of diseases which disrupt erythrocytes, based on experimental reports that heptoglobin level in blood serum from a malaria patient is lower than a normal.

Nevertheless, since heptoglobin level in blood serum has been measured by using polyclonal antibody, the prior art method reveals a shortcoming that it detects monomer, dimer, and degraded fragments as well, leading to a wide range of errors than the method of counting the numbers of erythrocytes, therefore, it is no more used in the art. If normal heptoglobin level in blood serum can be measured specifically, the diagnosis of diseases which disrupt erythrocytes would be made in an accurate manner.

Under the circumstances, there are strong reasons for exploring and developing a method for measuring normal heptoglobin level in blood serum.

### DISCLOSURE OF INVENTION

### Technical Problem

The present inventors have made an effort to develop a method for measuring normal heptoglobin level in blood serum, and discovered that only normal heptoglobin level can be measured to diagnose an attack of diseases which disrupt erythrocytes can be diagnosed in an accurate manner, by using a kit for the immunodetection of heptoglobin comprising antibodies which bind specifically to α and β subunits of dimeric heptoglobin, respectively.

### Technical Solution

The first object of the invention is, therefore, to provide a method for measuring normal heptoglobin level in blood serum by using antibodies which bind specifically to α and β subunits of heptoglobin.

The second object of the invention is to provide a method for diagnosing an attack of diseases which disrupt erythrocytes by using antibodies which bind specifically to α and β subunits of heptoglobin.

The third object of the invention is to provide a kit for immunodetection of heptoglobin which measures heptoglobin level in blood serum.

The fourth object of the invention is to provide a method for diagnosing an attack of diseases which disrupt erythrocytes by using the kit for immunodetection of heptoglobin.

### BRIEF DESCRIPTION OF THE DRAWINGS

The above and the other objects and features of the present invention will become apparent from the following descriptions given in conjunction with the accompanying drawings, in which:

FIG. 1 is a photograph showing an electrophoresis pattern of proteins in blood serum from a normal.

FIG. 2 is a photograph showing an electrophoresis pattern of proteins in blood serum from a malaria patient.

### BEST MODE FOR CARRYING OUT THE INVENTION

The present inventors performed 2D-electrophoresis for the blood sera from a normal and a malaria patient and compared protein patterns with each other, which revealed that hemoglobin level increased but heptoglobin level decreased in blood serum of a malaria patient. They also measured heptoglobin levels in blood sera from a normal and a malaria patient by using antibodies which bind specifically to α and β subunits of heptoglobin, respectively, and by using an antibody for heptoglobin, and comparison of them with each other was followed. As a result, they found that the levels of dimeric, monomeric and degraded heptoglobins were totally measured by using the antibody for a dimeric heptoglobin, whereas only the level of normal heptoglobin was measured by using the antibodies for α and β subunits of heptoglobin, implying that the level of dimeric heptoglobin can be measured specifically by using antibodies which bind specifically to α and β subunits of heptoglobin, respectively.

A method for measuring heptoglobin level in blood serum of the present invention comprises the steps of: (i) reacting blood serum of a subject with an antibody (αh antibody) which binds specifically to α subunit of a dimeric heptoglobin and an antibody (βh antibody) which binds specifically to β subunit of a dimeric heptoglobin; and, (ii) measuring level of proteins which react with the ah antibody and βh antibody in blood serum and have a molecular weight of more than 100 kDa. The level of proteins, not limited thereto, is preferably measured by Western blot analysis or Sandwich ELISA method using a pair of antibodies: a non-labeled αh antibody and a labeled βh antibody; or, a labeled αh antibody and a non-labeled βh antibody. The labeling of antibody is done by: fluorescent materials such as Cy-3, Cy-5, FITC (fluorescein isothiocyanate), GFP (green fluorescent protein), RFP (red fluorescent protein) or Texas Red; radioisotopes such as ³H, ¹⁴C or ³²P; or, enzymes such as HRP (horse raddish peroxidase), alkaline phosphatase, β-galactosidase or luciferase.

According to the method for measuring normal heptoglobin level in blood serum, the present inventors demonstrated that an attack of disease such as a malaria which disrupts erythrocyte can be diagnosed by measuring the normal heptoglobin level in blood serum which employs antibodies which bind specifically to α and β subunits of heptoglobin, respectively, or by using a kit for immunodetection of heptoglobin containing the said antibodies.

The present invention provides a method for diagnosing an attack of disease which disrupts erythrocyte, which comprises the steps of: (i) reacting blood serum of a subject with an antibody (αh antibody) which binds specifically to α subunit of a dimeric heptoglobin and an antibody (βh antibody) which binds specifically to β subunit of a dimeric heptoglobin; (ii) measuring level of proteins which react with the ah antibody and βh antibody in blood serum and have a molecular weight of more than 100 kDa; and, comparing the measured protein level with protein level of a normal, where the level of proteins is measured in a similar manner as described above.

Further, the present invention provides a kit for immunodetection of heptoglobin containing antibodies which bind specifically to α and β subunits of a dimeric heptoglobin, respectively, which comprises: (i) a plate on which an antibody which binds specifically to α subunit or β subunit of heptoglobin is fixed; (ii) a solution containing an antibody which binds specifically to α subunit or β subunit which is not bound to the antibody fixed on the plate; (iii) a secondary antibody which binds to the antibody in the solution; and, (iv) a means for detecting the secondary antibody. The plate, not limited thereto, is preferably glass or plastic, the secondary antibody, not limited thereto, is preferably labeled with biotin- or peroxidase-labeled rabbit-goat serum, and the means for detecting the secondary antibody, not limited thereto, is preferably hydrogen peroxide or streptavidin.

Finally, the present invention provides a method for diagnosing an attack of disease such as a malaria which disrupts erythrocyte by measuring dimeric heptoglobin level by using the kit for immunodetection of heptoglobin. The method for diagnosing an attack of disease which disrupts erythrocyte comprises the steps of: (i) collecting blood sera from a normal and a subject who is doubted as a patient suffering from a disease which disrupts erythrocyte; (ii) adding each of the collected sera to a plate in the said kit to react with an antibody fixed on the plate; (iii) removing the serum from the plate and adding a solution containing an antibody in the said kit to the plate; (iv) removing the solution from the plate and adding a secondary antibody in the said kit; (v) removing the secondary antibody from the plate and adding a means for detecting the secondary antibody in the said kit to measure levels of heptoglobin; and, (vi) comparing the levels of heptoglobin with each other. The secondary antibody and means for detecting the secondary antibody are the same as described above.

### MODE FOR THE INVENTION

The present invention is further illustrated in the following examples, which should not be taken to limit the scope of the invention.

### EXAMPLE 1

### Comparison of Levels of Proteins in Blood Sera from a Normal and a Malaria Patient

2D-electrophoresis for the blood sera from a normal and a malaria patient was performed as follows. Blood serum was collected from a normal and a malaria patient, respectively, and blood serum containing 150□ of serum proteins was mixed with 50 □ of SDS/DTT solubilization buffer (0.3% SDS, 3% DTT (dithiothreitol), 50 mM Tris/HCl, pH 8.0), and incubated at 95° C. for 5 min. Then, 500 □ of enhanced solubilising solution (7 M urea, 2 M thiourea, 4% CHAPS (3-[(3-cholamidopropyl)dimethylammonio]-1-propan-sulfonate), 40 mM Tris/HCl, 0.002% bromophenol blue, 2 mM tributylphospine) was added and centrifuged at 17,000 rpm for 15 min, at 4° C. to obtain a supernatant. And then, 500 □ of the supernatant was loaded on immobiline pH gradient (IPG) dry strip tray and the IPG dry strip (pH 3-10, 18□) was rehydrated for 16 hours. IEF (isoelectric focusing) of the rehydrated strip was performed by applying a voltage of 100 kVh in Multiphor apparatus (Amersham-Pharmacia Biotech, Sweden) to move serum proteins to isoelectric point (PI).

90 ml of 5× Tris/HCl (1.875 M, pH 8.8), 90 ml of 40% acrylamide solution, 4.5 ml of 10% SDS, 90□ of 100 mM sodium thiosulfate, 740 □ of 10% ammonium persulfate, 74□ of TEMED and 270 ml of distilled water were mixed to prepare 8% polyacrylamide gradient gel solution, and 90 ml of 5× Tris/HCl (1.875 M, pH 8.8), 180 ml of 40% acrylamide solution, 90 ml of 50% glycerol, 4.5 ml of 10% SDS, 90□ of 100 mM sodium thiosulfide, 740□ of 10% ammonium persulfate, 74□ of TEMED and 90 ml of distilled water were mixed to prepare 16% polyacrylamide gradient gel solution. Then, 8 to 16% gradient gel SDS-PAGE (18 cm width×1.5 mm height) was prepared by using the polyacrylamide gel solutions prepared as above.

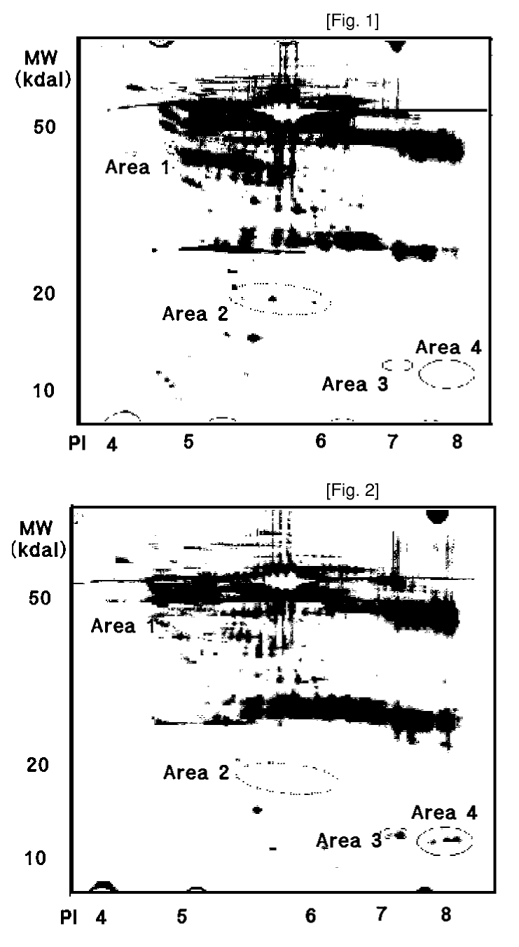

To the IEF performed strip was added 10 ml of TBP equilibrium buffer solution (0.2 mM tributyl phosphine, 6 M urea, 2% SDS, 375 mM Tris buffer (pH 8.8), 20% glycerol and 2.5% acrylamide), and shaked slightly for 15 min to reach an equilibrium state. The equilibrated strip was loaded on the SDS-PAGE gel prepared as above, and poured agarose embedding solution (agarose, 1% bromophenol blue, 1×SDS running buffer) up to the glass top to fix the strip on the gel. The strip on 8-16% gradient gel was put into a bottle containing SDS-PAGE running buffer (glycine, SDS and Tris-base), and electrophoresed in 200 mA for 12 hours. Then, gel was stained to compare protein level of blood serum from a normal with that from a malaria patient (see: FIGS. 1 and 2). FIGS. 1 and 2 are photographs showing an electrophoresis pattern of proteins in blood sera from a normal and a malaria patient, respectively. As shown in FIGS. 1 and 2, it was demonstrated that proteins in blood serum from a malaria patient in Areas 1 and 2 were significantly decreased, whereas proteins in Areas 3 and 4 were significantly increased.

Proteins from each area were isolated and treated with trypsin, and subjected to MALDI-TOF analysis, which revealed that all proteins in Areas 1 and 2 were identified as heptoglobin, proteins in Areas 3 and 4 were identified as hemoglobin, respectively. Accordingly, it was clearly demonstrated that heptoglobin level in blood serum from a malaria patient is lower than that of a normal.

### EXAMPLE 2

### Preparation of a Kit for Immunodetection of Heptoglobin

Antibodies for α and β subunits of heptoglobin were prepared, respectively, and employed to prepare a kit for immunodetection of heptoglobin as follows.

### EXAMPLE 2-1

### Preparation of Antibodies for α and β Subunits of Heptoglobin

A mixture of 2 ml of α subunit of heptoglobin and 2 ml of adjuvant was injected into 7 weeks old rabbit three times at an interval of 1 week and two times at an interval of 2 weeks. Then, small amount of blood was collected from the immunized rabbit and measured titer of an antibody for α subunit, and whole blood was collected from the immunized rabbit by cardiac puncture. The blood thus collected was left to stand for 1 day at room temperature to obtain blood serum as a supernatant. And then, the blood serum was electrophoresed on 12% (v/v) acrylamide gradient gel, and subjected to Western blot analysis using α subunit to determine molecular weight of antibodies in blood serum.

Then, the blood serum was applied on a gel filtration chromatography which is capable of separating proteins in a range of molecular weight determined as above, fractions were collected and subjected to Western blotting to identify fractions containing an antibody for α subunit of heptoglobin. An antibody for β subunit of heptoglobin was also prepared similarly as the above except for using β subunit instead of α subunit.

### EXAMPLE 2-2

### Preparation of a Kit for Immunodetection of Heptoglobin

A glass plate on which an antibody for α subunit of heptoglobin is fixed, Tris buffer solution (50 mM Tris/HCl, pH 8.0) containing an antibody for β subunit of heptoglobin and 50% glycerol, peroxidase-labeled rabbit-goat serum (a secondary antibody) and hydrogen peroxide were prepared, respectively, to manufacture a kit for immunodetection of heptoglobin.

### EXAMPLE 3

### Comparison of Heptoglobin Levels in Blood Sera from a Normal and a Malaria Patient by Using a Kit for Immunodetection of Heptoglobin

To a plate of an immunodetection kit for heptoglobin prepared in Example 2 was added each of blood sera collected from a normal and a malaria patient prepared in Example 1, respectively, incubated for 2 hours at room temperature, removed the blood serum from the plate, and washed with PBS. Then, to the plate was added a solution containing an antibody which binds specifically to α or β subunit of heptoglobin, incubated for 2 hours at room temperature, removed the solution from the plate, and washed with PBS again. And then, peroxidase-labeled rabbit-goat serum of the kit was added to the plate, reacted for 30 min at room temperature, added hydrogen peroxide, and measurement of absorbance was followed in ELISA reader.

### COMPARATIVE EXAMPLE

### Comparison of Heptoglobin Levels in Blood Sera from a Normal and a Malaria Patient by Using an Antibody for Heptoglobin

Measurement of heptoglobin level in blood sera was performed by using an antibody for dimeric heptoglobin, not antibodies for α and β subunits of heptoglobin:

An antibody for heptoglobin was prepared in a similar manner as in Example 2-1 except for using a dimeric heptoglobin instead of α subunit. Then, a glass plate on which an antibody for dimeric heptoglobin is fixed, Tris buffer solution (50 mM Tris/HCl, pH 8.0) containing an antibody for dimeric heptoglobin and 50% glycerol, peroxidase-labeled rabbit-goat serum (a secondary antibody) and hydrogen peroxide were prepared, respectively, to manufacture a kit for immunodetection of dimeric heptoglobin. And then, absorbance measurement for blood sera from a normal and a malaria patient was followed in a similar manner as in Example 3, except for using a kit for immunodetection of dimeric heptoglobin, and compared with the results of Example 3 (see: Table 1)

TABLE 1
Comparison of the heptoglobin levels in blood
sera from a normal and a malaria patient

| | Blood serum of | Blood serum of malaria |
|---|---|---|
| | normal | patient |
| Example 3 | 1.25 | 0.12 |
| Comparative Example | 2.01 | 0.94 |

(unit: absorbance)

As can be seen in Table 1 above, it was found that heptoglobin level measured by using a kit of heptoglobin comprising antibodies for α and β subunits of heptoglobin (Example 3) is lower than that measured by using a kit comprising an antibody for dimeric heptoglobin (Comparative Example), implying that the levels of dimeric, monomeric and degraded heptoglobins were totally measured by using the antibody for dimeric heptoglobin (Comparative Example), whereas only the level of normal heptoglobin was measured by using the antibodies for α and β subunits of heptoglobin (Example 3). Therefore, it was clearly demonstrated that the level of heptoglobin in blood serum can be measured accurately by employing an immunodetection kit for heptoglobin of present invention.

### INDUSTRIAL APPLICABILITY

As clearly explained and demonstrated as the above, the present invention provides a method for measuring heptoglobin level in blood serum by using antibodies for α and β subunits of heptoglobin, and a kit for immunodetection of heptoglobin which comprises the antibodies for α and β subunits of heptoglobin for measuring heptoglobin level in blood serum. The method for measuring heptoglobin level in blood serum can be practically applied for the early diagnosis of diseases which disrupt erythrocytes, since only the level of normal heptoglobin except for monomeric and degraded heptoglobins can be specifically measured by the said method.

1. A method for measuring heptoglobin level in blood serum, which comprises the steps of:
(i) reacting blood serum of a subject with an antibody (αh antibody) which binds specifically to α subunit of a dimeric heptoglobin and an antibody (βh antibody) which binds specifically to β subunit of a dimeric heptoglobin; and,
(ii) measuring level of proteins which react with the αh antibody and βh antibody in blood serum and have a molecular weight of more than 100 kDa.

2. The method for measuring heptoglobin level in blood serum of claim 1, wherein the level of proteins is measured by Western blot analysis.

3. The method for measuring heptoglobin level in blood serum of claim 1, wherein the level of proteins is measured by Sandwich ELISA method.

4. The method for measuring heptoglobin level in blood serum of claim 3, wherein the level of proteins is measured by using a labeled αh antibody and a non-labeled βh antibody.

5. The method lee measuring heptoglobin level in blood serum of claim 3, wherein the level of proteins is measured by using a non-labeled αh antibody and a labeled βh antibody.

6. The method for measuring heptoglobin level in blood serum of claim 4, wherein the labeling of antibody is done by fluorescent material, radioisotope or enzyme.

7. The method for measuring heptoglobin level in blood serum of claim 6, wherein the fluorescent material is Cy-3, Cy-5, FITC (fluorescein isothiocyanate), GFP (green fluorescent protein), RFP (red fluorescent protein) or Texas Red.

8. The method for measuring heptoglobin level in blood serum of claim 6, wherein the enzyme is HRP (horse raddish peroxidase), alkaline phosphatase, beta-galactosidase or luciferase.

9. A method for diagnosing an attack of disease which disrupts erythrocyte, which comprises the steps of:
reacting blood serum of a subject with an antibody (αh antibody) which binds specifically to α subunit of a dimeric heptoglobin and an antibody (βh antibody which binds specifically to β subunit of a dimeric heptoglobin;
(ii) measuring level of proteins which react with the αh antibody and βh antibody in blood serum and have a molecular weight of more than 100 kDa; and,
(iii) comparing the measured protein level with protein level of a normal.

10. The method for diagnosing an attack of disease which disrupts erythrocyte of claim 9, wherein the level of proteins is measured by Western blot analysis.

11. The method for diagnosing an attack of disease which disrupts erythrocyte of claim 9, wherein the level of proteins is measured by Sandwich ELISA method.

12. The method for diagnosing an attack of disease which disrupts erythrocyte of claim 11, wherein the level of proteins is measured by using a labeled αh antibody and a non-labeled βh antibody.

13. The method for diagnosing an attack of disease which disrupts erythrocyte of claim 11, wherein the level of proteins is measured by using a non-labeled αh antibody and a labeled βh antibody.

14. The method for diagnosing an attack of disease which disrupts erythrocyte of claim 12, wherein the labeling of antibody is done by fluorescent material, radioisotope or enzyme.

15. The method for diagnosing an attack of disease which disrupts erythrocyte of claim 14, wherein the fluorescent material is Cy-3, Cy-5, FITC (fluorescein isothiocyanate), GFP (green fluorescent protein), RFP (red fluorescent protein) or Texas Red.

16. The method for diagnosing an attack of disease which disrupts erythrocyte of claim 14, wherein the enzyme is HRP (horse raddish peroxidase), alkaline phosphatase, beta-galactosidase or luciferase.

17. The method for diagnosing an attack of disease which disrupts erythrocyte of claim 9, wherein the disease is malaria.

18. A kit for immunodetection of heptoglobin which comprises;
(i) a plate on which an antibody which binds specifically to α subunit or β subunit of heptoglobin is fixed;
(ii) a solution containing an antibody which binds specifically to α subunit or β subunit which is not bound to the antibody on the plate;
(iii) a secondary antibody which binds to the antibody in the solution; and,
(iv) a means for detecting the secondary antibody.

19. The kit for immunodetection of heptoglobin of claim 18, wherein the plate is glass or plastic.

20. The kit for immunodetection of heptoglobin of claim 18, wherein the secondary antibody is labeled with biotin- or peroxidase-labeled rabbit-goat serum.

21. The kit for immunodetection of heptoglobin of claim 18, wherein the means for detecting the secondary antibody is hydrogen peroxide or streptavidin.

22. A method for diagnosing an attack of disease which disrupts erythrocyte by using a kit for immunodetection of heptoglobin of claim 18, which comprises the steps of:
(i) collecting blood sera from a normal and a subject who is doubted as a patient suffering from a disease which disrupts erythrocyte;
(ii) adding each of the collected sera to a plate in the said kit to react with an antibody fixed on the plate;
(iii) removing the serum from the plate and adding a solution containing an antibody in the said kit to the plate;
(iv) removing the solution from the plate and adding a secondary antibody in the said kit;
(v) removing the secondary antibody from the plate and adding a means for detecting the secondary antibody in the said kit to measure levels of heptoglobin; and,
(vi) comparing the levels of dimeric heptoglobin with each other.

23. The method for diagnosing an attack of disease which disrupts erythrocyte of claim 22, wherein the disease is malaria.

24. The method for diagnosing an attack of disease which disrupts erythrocyte of claim 22, wherein the secondary antibody is labeled with biotin- or peroxidase-labeled rabbit-goat serum.

25. The method for diagnosing an attack of disease which disrupts erythrocyte of claim 22, wherein the means for detecting the secondary antibody is hydrogen peroxide or streptavidin.

26. A kit for diagnosing malaria, which comprises:
(i) a plate on which an antibody which binds specifically to α subunit or β subunit of heptoglobin is fixed;
(ii) a solution containing an antibody which binds specifically to α subunit or β subunit which is not bound to the antibody fixed on the plate;
(iii) biotin- or peroxidase-labeled secondary antibody which binds to the antibody in the solution; and,
(iv) hydrogen peroxide or streptavidin for detecting the secondary antibody.

27. The method for measuring heptoglobin level in blood serum of claim 5, wherein the labeling of antibody is done by fluorescent material, radioisotope or enzyme.

28. The method for diagnosing an attack of disease which disrupts erythrocyte of claim 13, wherein the labeling of antibody is done by fluorescent material, radioisotope or enzyme.
